(19) United States
(12) Patent Application Publication
(10) Pub. No.: US 2005/0054716 A1
(43) Pub. Date: Mar. 10, 2005

(54) Pharmaceutical compositions and methods of using taxane derivatives

(76) Inventors: Uday Shankar Gogate, North Brunswick, NJ (US); Deborah S. Piperno, Lincroft, NJ (US); Robert Kevin Perrone, Belle Mead, NJ (US); Krishnaswamy Srinivas Raghavan, Cranbury, NJ (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 10/704,401

(22) Filed: Nov. 7, 2003

Related U.S. Application Data
(60) Provisional application No. 60/424,848, filed on Nov. 8, 2002.

(51) Int. Cl. A61K 31/337

(52) U.S. Cl. 514/449

(57) ABSTRACT

The present invention relates to a novel intravenous formulation for a taxane chemotherapeutic agent. The agent is formulated with ethanol, polyoxyethylated castor oil and a mixture of antioxidants to prevent oxidation of the drug substance caused by the polyoxyethylated castor oil.

### FIELD OF THE INVENTION

This application claims priority benefit under Title 35 § 119(e) of U.S. provisional Application No. 60/424,848 filed Nov. 8, 2002.

The present invention relates to a novel formulation for taxane compounds, said formulation characterized by increased solubility and stability, and resistance to oxidation.

### BACKGROUND OF THE INVENTION

U.S. Pat. No. 5,646,176 discloses taxane derivatives and their use as anti-tumor agents. The compounds disclosed herein have been found useful for the treatment of certain types of cancer including bladder and gastric cancer.

Taxol® (paclitaxel) is a natural product extracted from the bark of Pacific yew trees, Taxus brevifolia. It has been shown to have excellent antitumor activity in in vivo animal models, and recent studies have elucidated its unique mode of action, which involves abnormal polymerization of tubulin and disruption of mitosis. It has recently been approved for the treatment of refractory advanced ovarian cancer and breast cancer; and studies involving other cancers have shown promising results. The results of paclitaxel clinical studies are reviewed by numerous authors, such as by Rowinsky and Donehower in “The Clinical Pharmacology and Use of Antimicrotubule Agents in Cancer Chemotherapeutics”, Pharmac. Ther., 52:35-84, 1991; by Spencer and Faulds in “Paclitaxel, A Review of its Pharmacodynamic and Pharmacokinetic Properties and Therapeutic Potential in the Treatment of Cancer”, Drugs, 48 (5) 794-847, 1994; by K. C. Nicolaou et al. in “Chemistry and Biology of Taxol”, Angew. Chem., Int. Ed.Engl., 33: 15-44, 1994; by F. A. Holmes, A. P. Kudelka, J. J. Kavanaugh, M. H. Huber, J. A. Ajani, V. Valero in the book “Taxane Anticancer Agents Basic Science and Current Status” edited by Gunda I. Georg, Thomas T. Chen, Iwao Ojima, and Dolotrai M. Vyas, 1995, American Chemical Society, Washington, D.C., 31-57; by Susan G. Arbuck and Barbara Blaylock in the book “TAXOL® Science and Applications” edited by Mathew Suffness, 1995, CRC Press Inc., Boca Raton, Fla., 379-416; and also in the references cited therein.

Derivatives of Taxol® have been found to possess antitumor activity; however, it has been challenging to prepare formulations of these derivatives because of their inherent insolubility and their susceptibility to oxidation when used with standard formulations of Taxol® containing polyoxyethylated (POE) castor oil and other carriers.

### SUMMARY OF THE INVENTION

The present invention is directed to a novel formulation which comprises
a) at least one taxane compound of the formula 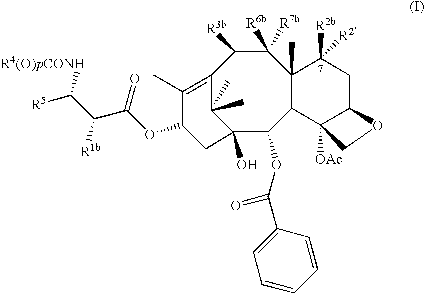
   wherein:
R^{1b} is hydroxy, protected hydroxy, —OCH₂SCH₃, —OC(O)R^{x} or —OC(O)OR^{x};
R² is hydrogen, and
R^{2b} is hydrogen, hydroxy, protected hydroxy, —OCH₂SCH₃ or —OC(O)OR^{x};
R^{3b} is hydrogen, hydroxy, protected hydroxy, C₁₋₆ alkyloxy, —OC(O)R^{x}, —OCH₂SCH₃ or —OC(O)OR^{x}; one of R^{6b} or R^{7b} is hydrogen and the other is hydroxy, protected hydroxy, C₁₋₆ alkanoyloxy or —OCH₂SCH₃; or R^{6b} and R^{7b} together form an oxo group; with the proviso that at least one of R^{1b}, R^{2b}, R^{3b}, R^{6b} or R^{7b} is —OCH₂SCH₃;
p is 0 or 1;
R^{x} is a radical of the formula 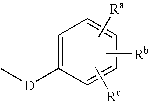
   wherein
D is a bond or C₁₋₆ alkyl; and
R^{a}, R^{b} and R^{c} are independently hydrogen, amino C₁₋₆ alkylamino, di-C₁₋₆ alkylamino, halogen, C₁₋₆ alkyl, or C₁₋₆ alkoxy; R⁴ and R⁵ are independently C₁₋₆ alkyl, C₂₋₆ alkenyl, C₂₋₆alkynyl, or —ZR⁶; wherein
Z is a direct bond, C₁₋₆ alkyl or C₂₋₆ alkenyl; and
R⁶ is aryl, substituted aryl, C₃₋₆ cycloalkyl, or heteroaryl;
b) in a suitable mixture of solvents;
c) in a pharmaceutically effective amount of a buffer, and
d) containing a mixture of antioxidants.

In a preferred embodiment, the formulation of the invention employs compound Ia of the formula 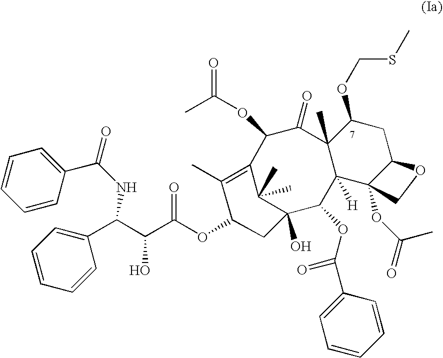
with the above described substituents.

The compounds represented by formula (I) are novel compounds that are useful in the treatment of a variety of cancers and other abnormal proliferative diseases. The novel formulation increases the solubility and stability of the insoluble compounds and provides for the use of antioxidants to prevent degradation of the chemotherapeutic agent.

The invention also provides methods for their use in the treatment of cancer.

### DETAILED DESCRIPTION OF THE INVENTION

The present invention is directed to a novel formulation which comprises
a) a pharmaceutically effective amount of at least one taxane compound of the formula 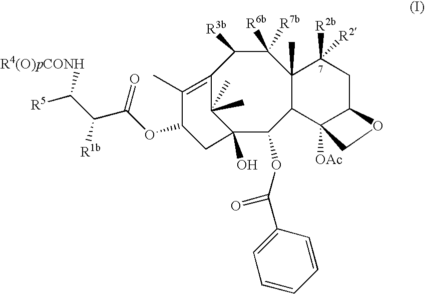
   wherein:
R^{1b} is hydroxy, protected hydroxy, —OCH₂SCH₃, —OC(O)R^{x} or —OC(O)OR^{x};
R is hydrogen, and
R^{2b} is hydrogen, hydroxy, protected hydroxy, —OCH₂SCH₃ or —OC(O)OR^{x};
R^{3b} is hydrogen, hydroxy, protected hydroxy, C₁₋₆ alkyloxy, —OC(O)R^{x}, —OCH₂SCH₃ or —OC(O)OR^{x}; one of R^{6b} or R^{7b} is hydrogen and the other is hydroxy, protected hydroxy, C₁₋₆ alkanoyloxy or —OCH₂SCH₃; or
R^{6b} and R^{7b} together form an oxo group; with the proviso that at least one of R^{1b}, R^{2b}, R^{3b}, R^{6b} or R^{7b} is —OCH₂SCH₃;
p is 0 or 1;
R^{x} is a radical of the formula 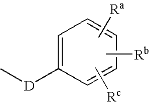
   wherein
D is a bond or C₁₋₆ alkyl; and
R^{a}, R^{b} and R^{c} are independently hydrogen, amino C₁₋₆ alkylamino, di-C₁₋₆ alkylamino, halogen, C₁₋₆ alkyl, or C₁₋₆ alkoxy;
R⁴ and R⁵ are independently C₁₋₆ alkyl, C₂₋₆ alkenyl, C₂₋₆alkynyl, or —ZR⁶; wherein Z is a direct bond, C₁₋₆ alkyl or C₂₋₆ alkenyl; and
R⁶ is aryl, substituted aryl, C₃₋₆ cycloalkyl, or heteroaryl;
b) in a suitable mixture of solvents;
c) in a pharmaceutically effective amount of a buffer, and
d) containing a mixture of antioxidants.

In a preferred embodiment, the compound of formula I is the compound of formula Ia shown below, which is 7-O-methylthiomethylpaclitaxel 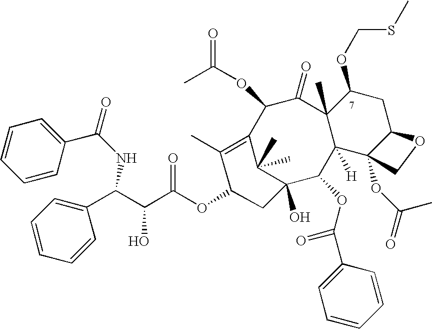
with the above described substituents.

Listed below are definitions of various terms used to describe this invention. These definitions apply to the terms as they are used throughout this specification, unless otherwise indicated in specific instances.

“Alkyl” means a straight or branched saturated carbon chain having from one to six carbon atoms; examples include methyl, ethyl, n-propyl, isopropyl, n-butyl, sec-butyl, isobutyl, t-butyl, n-pentyl, sec-pentyl, isopentyl, and n-hexyl.

“Alkenyl” means a straight or branched carbon chain having at least one carbon-carbon double bond, and having from two to six carbon atoms; examples include ethenyl, propenyl, isopropenyl, butenyl, isobutenyl, pentenyl, and hexenyl.

“Alkynyl” means a straight or branched carbon chain having at least one carbon-carbon triple bond, and from two to six carbon atoms; examples include ethynyl, propynyl, butynyl, and hexynyl.

“Aryl” means aromatic hydrocarbon having from six to ten carbon atoms; examples include phenyl and naphthyl. “Substituted aryl” means aryl substituted with at least one group selected from C₁₋₆ alkanoyloxy, hydroxy, halogen, C₁₋₆ alkyl, trifluoromethyl, C₁₋₆ alkoxy, aryl, C₂₋₆ alkenyl, C₁₋₆ alkanoyl, nitro, amino, and amido.

“Halogen” means fluorine, chlorine, bromine, and iodine.

“Taxane derivative” refers to a compound having a taxane moiety bearing a C₁₃ sidechain.

“Heteroaryl” means a five- or six-membered aromatic ring containing at least one and up to four non-carbon atoms selected from oxygen, sulfur and nitrogen. Examples of heteroaryl include thienyl, furyl, pyrrolyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, triazinyl, tetrazinyl, and like rings.

“Hydroxy protecting groups” include, but is not limited to, ethers such as methyl, t-butyl, benzyl, p-methoxybenzyl, p-nitrobenzyl, allyl, trityl, methoxymethyl, methoxyethoxymethyl, ethoxyethyl, tetrahydropyranyl, tetrahydrothiopyranyl, and trialkylsilyl ethers such as trimethylsilyl ether, triethylsilyl ether, and t-butyldimethylsilyl ether; esters such as benzoyl, acetyl, phenylacetyl, formyl, mono-, di-, and trihaloacetyl such as chloroacetyl, dichloroacetyl, trichloroacetyl, trifluoroacetyl; and carbonates such as methyl, ethyl, 2,2,2-trichloroethyl, allyl, benzyl, and p-nitrophenyl.

Additional examples of hydroxy protecting groups may be found in standard reference works such as Greene and Wuts, Protective Groups in Organic Synthesis, 2d Ed., 1991, John Wiley & Sons, and McOmie, Protective Groups in Organic Chemistry, 1975, Plenum Press. Methods for introducing and removing protecting groups are also found in such textbooks.

The term “container” means any pharmaceutically acceptable vessel that could be used to hold a liquid solution and that is amenable to the administration of an intravenous or intramuscular formulation. These include vials, sterile bags, syringes and the like.

The formulation of the present invention provides an advantageous method for the administration of the compound by increasing the solubility, decreasing the oxidation of and maintaining drug stability during shelf-life storage and following aqueous dilution.

The compounds of the invention are microtubule-stabilizing agents and, thus, can be used to treat a variety of cancers or other diseases of abnormal cell proliferation. The methods of the invention are particularly useful for administering the compounds of the invention to a patient suffering from cancer or other hyperproliferative cellular disease. As used herein, the term “cancer” includes, but is not limited to, solid tumors and blood born tumors. The term cancer refers to disease of skin, tissues, organs, bone, cartilage, blood and vessels. The term “cancer” further encompasses primary and metastatic cancers.

Compound Ia by itself has low intrinsic aqueous solubility (<0.1 μg/ml) and a salt formation could not be used since the compound does not ionize in a desirable physiological pH range. Therefore, it was necessary to formulate the compound in such a way to get the desired solubility at a physiological pH and maintain stability prior to administration. It was determined that while the solubility is higher in solvents other than water, drug precipitation occurs upon aqueous dilution.

It is expected that the daily human dose is approximately 120 mg. In order to achieve a practical volume of infusion, a solution with higher drug concentration (than 0.1 μg/ml aqueous solubility) is required.

Various co-solvents were evaluated for solubility enhancement. Preferred solvents of the invention include ethanol, t-butyl alcohol, propylene glycol, glycerin, benzyl benzoate and N,N-dimethylacetamide. Particularly preferred are ethanol and t-butyl alcohol and these were further studied. It was discovered that 75% v/v ethanol (dehydrated alcohol) in water for injection provided the highest solubility of the preferred compounds at >17.5 mg/mL. A drug concentration of 15 mg of compound/mL in the 75% v/v ethanol:water was selected for further study.

It was also determined that the composition should include a buffer to help stability. Preferred buffering agents include citrate, tartrate, fumarate, oxalate, benzoate, acetate, succinate or lactate buffers, with the tartrate particularly preferred.

The 15 mg/mL solution included a 10 mM tartrate buffer which provided adequate solubility and stability, however, the solution could not be injected directly into patients because the non-aqueous components amounted to greater than 20% which potentially causes irritation at the injection site. Further dilution of this solution is therefore required.

Various diluents such as sodium chloride solution and dextrose injection were tried but both resulted in drug precipitation. Polysorbate, polyethylene glycol and polyoxyethylated (POE) castor oil are the preferred co-solvents with POE castor oil particularly preferred Three solutions containing mixtures of POE castor oil with ethanol were prepared and analyzed for compound concentration. An aqueous solution containing 7.5% ethanol and 4% POE castor oil was selected.

While this solution solves the injection site irritation problem, it was noted that drug degradation occurs due to peroxide impurities in the POE castor oil. It was determined that the degradation pathway could be avoided by either separating the drug substance from the POE castor oil by utilizing a two-container system or by the use of an antioxidant or mixture of antioxidants.

A number of prototype formulations containing up to three antioxidants were evaluated for stability. The antioxidants included sodium formaldehyde sulfoxylate, ascorbic acid, monothioglycerol, L-cysteine HCl, sodium bisulfite, butylatedhydroxytoluene, propyl gallate and Vitamin E. In addition to antioxidants, each formulation contained 1.5 mg/mL of therapeutic agent, 0.075 mL/mL ethanol, 0.04 mL/mL POE castor oil, in tartrate buffer, sealed under a nitrogen atmosphere. The test demonstrated that stabilization of the solution from oxidation was best achieved by the inclusion of ascorbic acid, L-cysteine hydrochloride and sodium formaldehyde sulfoxylate at a number of levels tested. It was surprisingly found, however, that inclusion of one or two antioxidants did not provide adequate stabilization. Additionally, it was found that adequate stability in the presence of air upon long term storage was realized only at 0.1% or greater (w/v) of each of these three antioxidants.

A preferred composition is detailed below in Table VII.

The compositions of the invention are preferably provided in the form of unit doses in sealed vials, preferably glass vials, most preferably Type I glass vials closed with elastomer stoppers. The preferred unit dose will contain a pharmaceutically effective amount of a taxane derivative, together with ethanol and POE castor oil as cosolvents in an aqueous buffer containing a mixture of antioxidants.

By way of illustration, and without serving as limitations in any way, the following examples serve to illustrate the practice of the invention.

### EXAMPLES

The compound was subjected to early solubility studies, to determine which co-solvent could be used to increase drug solubility, according to the following procedure.

### Example 1

### Solubility of Compound in a Co-Solvent:Water Mixture

Approximately 25 mg of drug substance was added to 2 mL aqueous solution of ethanol (33%, 50% and 75% v/v). An additional 10 mg of drug substance was added to the 75% sample as all drug appeared to dissolve. A similar study was performed by adding approximately 25 mg drug substance to 2 mL aqueous solution of tertiary butyl alcohol (33%, 50% and 66% v/v). Samples were stirred for over 16 hours, filtered through 0.45μ nylon syringe filters, diluted and analyzed by HPLC for drug concentration. The results shown in Table I indicate that among the conditions evaluated, 75% v/v dehydrated alcohol in water provided the highest solubility. Based on the results of these studies, a formulation of 15 mg/mL drug substance in 75% v/v ethanol:water was selected for further studies.

TABLE I

| Vehicle | | Solubility |
|---|---|---|
| Co-Solvent | % v/v | (mg/mL) |
| Tertiary Butyl Alcohol | 33 | 0.19 |
| | 50 | 3.13 |
| | 66 | 12.95 |
| Dehydrated Alcohol, USP | 33 | 0.03 |
| | 50 | 1.64 |
| | 75 | >17.5 |

The effect of pH on the drug substance stability was also studied. The buffer pH providing maximum stability was determined by comparing the stability of prototype formulations of the drug substance. Initial experiments evaluated solutions containing 0.2 mg drug/mL in 16.7% v/v ethanol:0.1M citrate buffers. Relative area percents of drug peaks were evaluated following 2 days storage at 85° C. HPLC analysis demonstrated that the best stability was achieved at buffer 4.5. Subsequent experiments evaluated stability (1 mg drug/mL) in 75% v/v ethanol:0.01M tartrate buffer. Three mL aliquots of samples were dispensed into 5 cc Type I glass vials and closed with West 4405/50 20 mm stoppers. Percent drug substance remaining, impurities and degradants were evaluated following 18 days storage at 50° C. and compared to initial values. A solution with apparent pH 5.4 (corresponding to tartrate buffer pH 3.8), was observed to be most stable. Based on these results, tartrate buffer pH 3.8 was selected for further experiment because the pH of maximum stability is within the buffering range of tartaric acid (pKₐ₁=3.02, pKₐ₂=4.54).

TABLE II

| | | | Total |
|---|---|---|---|
| | | | Impurity |
| | | % Compound | Index |
| Buffer pH | Apparent pH | Remaining | (Area %) |
| 2.6 | 3.88 | 94.4 | 4.16 |
| 3.0 | 4.67 | 97.1 | 2.51 |
| 3.5 | 5.15 | 97.1 | 2.06 |
| 3.6 | 5.20 | 98.1 | 1.36 |
| 3.8 | 5.39 | 101 | 1.34 |
| 4.0 | 5.81 | 98.1 | 1.99 |
| 4.0 | 5.69 | 100 | 1.62 |
| 4.2 | 5.99 | 99 | 1.67 |
| 4.4 | 6.24 | 100 | 1.84 |

Drug solution (15 mg/mL) in 75% ethanol/10 mM tartrate buffer (apparent pH 5.4) was found to provide adequate solubility and stability. However, this solution cannot be injected directly into patients as the non-aqueous components exceed 20%, thus potentially causing irritation at the injection site. Dilution of this solution with aqueous diluents such as 0.9% sodium chloride injection or 5% dextrose injection causes drug precipitation. It has been shown that the precipitation can be avoided by inclusion of a co-solvent such as polyoxyethylated (POE) castor oil in the formulation. Subsequently, solubility of the drug substance was determined in solutions containing various amounts of dehydrated alcohol and POE castor oil. Approximately 20 mg of drug was added to 3 mL aliquots of the solutions shown below in Table III. Samples were stirred for 16 hours, filtered trough 0.45 micron nylon syringe filters and analyzed by HPLC for drug concentration. Results in Table III indicate that an aqueous solution containing 7.5% dehydrated alcohol and 4% POE castor oil provides adequate drug solubility (>1.5 mg/mL) with a minimized amount of co-solvent.

TABLE III

| Vehicle | | Solubility |
|---|---|---|
| Dehydrated Alcohol v/v | POE Castor Oil v/v | (mg/mL) |
| 9.375% | 5% | 2.91 |
| 7.50% | 4% | 2.38 |
| 3.75% | 2% | 0.96 |

However, as shown below in Table IV, drug degradation occurs in a solution containing ethanol and POE castor oil due to peroxide impurities present in POE castor oil.

TABLE IV

| | Potency | Total Impurities |
|---|---|---|
| Storage Conditions | (mg/mL) | (area %) |
| Initial | 2.2 | 2.1 |
| 6 days @ 50° C. | 2.0 | 10.9 |
| 16 days @ 50° C. | 1.9 | 16.5 |

This degradation pathway can be avoided by adding appropriate antioxidants, as disclosed herein or by separating the drug substance from POE castor oil via a two-container system as disclosed in a related application.

Table V shows the effect of the presence of POE castor oil on the stability of the injection solution containing ethanol and pH 5.4 tartrate buffer. As shown below, the stability of the solution containing POE castor oil was much lower than the injection solution without the co-solvent.

TABLE V

| | | % potency |
|---|---|---|
| Solution | Days Stored at 50° C. | remaining |
| with POE castor oil 4% | 16 | 86 |
| w/o POE castor oil | 28 | 100 |

A formulation containing 1.5 mg/mL of Compound 1a, 0.075 mL/mL of ethanol, 0.04 mL/mL of POE castor oil in a tartrate buffer and containing 0.1% each of sodium formaldehyde sulfoxylate, L-cysteine HCl and ascorbic acid was prepared. Samples were placed on stability at 25° C. for 4 months. During the 4 months of storage, there were no significant changes in appearance or pH of the samples. Additionally, there were no changes in potency or total oxidative degradants for the samples. Results of the study are shown in Table VI.

TABLE VI

| | | | | Oxidative |
|---|---|---|---|---|
| Storage | | | Potency | Degradants |
| Conditions | Appearance | pH | (mg/mL) | (Area %) |
| Initial | Clear, colorless | 3.71 | 1.44 | 0.11 |
| | solution | | | |
| 2 Month at 25° C. | Clear, colorless | 3.56 | 1.46 | 0.21 |
| | solution | | | |
| 4 Month at 25° C. | Clear, colorless | 3.57 | 1.48 | 0.12 |
| | solution | | | |

The following table details the preferred composition of the invention (60 mg/vial including a small overage for vial-needle syringe holdup:

TABLE VII

| | | Amount | | Amount | |
|---|---|---|---|---|---|
| Ingredient | Reason for Use | per mL | | per Vial | |
| Compound Ia | Active | 1.50 | mg | 61.4 | mg |
| | Ingredient | | | | |
| Dehydrated | Solvent | 0.075 | mL | 3.068 | mL |
| Alcohol, USP | | | | | |
| Purified POE | Solubilizer | 0.04 | mL | 1.636 | mL |
| Castor Oil | | | | | |
| Sodium Tartrate, | Stabilizer | 5.16 | mg | 211.2 | mg |
| Dihydrate | (buffer) | | | | |
| L-Cysteine HCl | Antioxidant | 1.11 | mg | 45.4 | mg |
| Monohydrate, USP | | | | | |
| Sodium | Antioxidant | 1.31 | mg | 53.6 | mg |
| Formaldehyde | | | | | |
| Sulfoxylate | | | | | |
| Dihydrate, NF | | | | | |
| Sodium Ascorbate, | Antioxidant | 1.12 | mg | 45.8 | mg |
| USP | | | | | |
| Water for Injection, | Solvent | q.s. to 1.0 | mL | q.s. to 40.9 | mL |
| USP | | | | | |

### Example 2

### Preparation of 7-O-methylthiomethylpaclitaxel (Compound 1a)

Benzoyl peroxide (0.98 g, 4 mmol) was added to a vigorously stirred mixture of paclitaxel (0.85 g, 1 mmol) and dimethyl sulfide (0.72 mL, 8 mmol) in dry acetonitrile (10 ml) at 0.degree. C. Stirring was continued for 2.5 hours at 0.degree. C. Progress of the reaction was monitored by silica gel TLC in toluene: acetone (2:1, v/v) solvent system (R_{f tax}.=0.38, R_{f prod.}=0.64), and when formation of higher mobility products was observed the reaction was quenched by evaporation of solvents using Rotavapor at 30.degree. C. A TLC analysis of the reaction mixture indicated the presence of some quantities of unreacted paclitaxel and 2′,7-O-bis(methylthiomethyl)paclitaxel. Separation of the title compound from the reaction mixture was achieved by flash column chromatography on Silica Gel 60 (40-63 .mu.m) EM Science (100 mL), column diameter: 2 in. using ethyl acetate:hexane (1:1, v/v) solvent system (R f prod. =0.34). The product (552 mg, 60% yield) was recovered from fractions 12 to 18 (each fraction ca. 20 ml).

MS (FAB/matrix NOBA, NaI, KI): [M+H].sup.+, m/z 914; [M+Na].sup.+, m/z 936; [M+K].sup.+, m/z 952

Elemental Analysis: C: 64.28 (calc. 64.39), H: 5.85 (calc. 6.07), N: 1.46 (calc. 1.53)

UV (MeOH): .λ.max=226 nm, E(1%/1 cm)=150, A=0.2653

IR (KBr): 3432, 3066, 2940, 1726, 1668, 1602, 1582, 1514, 1484, 1452, 1372, 1242, 1178, 1142, 1108, 1068, 1026, 990, 916, 884, 852, 802, 774, 710, 608, 570, 538, 482

. ¹H-NMR (CDCl₃) δ 1.15 (3H, s), 1.19 (3H, s), 1.73 (3H, s), 1.79 (H, s), 1.90 (3H, d), 2.09 (3H, S), 2.16 (3H, s), 2.29 (2H, d), 2.35 (3H, s), 2.77 (H, m), 3.70 (H, d), 3.83 (H, d), 4.17 (H, d), 4.26 (H, m, overlaps with H, d), 4.63 (2H, t), 4.77 (H, dd), 4.91 (H, d), 5.65 (H, d), 5.77 (H, dd), 6.16 (H, dd), 6.48 (H, s), 7.07 (H, d), 7.29-7.50 (10H, m), 7.57 (H, m), 7.73 (2H, d), 8.08 (2H, d).

The present invention also contemplates kits, for example, for inhibiting tumor growth comprising a container (such as a vial) containing a pharmaceutical formulation comprising a compound of the present invention, said compound in a pharmaceutically acceptable carrier.

The embodiments of the invention described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

1. A pharmaceutical composition for administration to a patient which comprises
a) a pharmaceutically effective amount of at least one compound of the formula 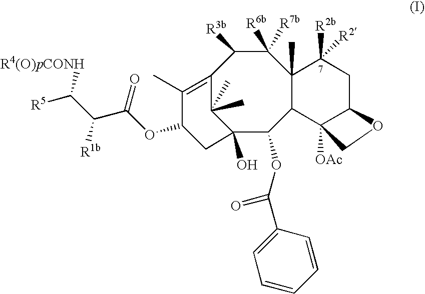
wherein:
R^{1b} is hydroxy, protected hydroxy, —OCH₂SCH₃, —OC(O)R^{x} or —OC(O)OR^{x};
R² is hydrogen, and
R^{2b} is hydrogen, hydroxy, protected hydroxy, —OCH₂SCH₃ or —OC(O)ORₓ;
R^{3b} is hydrogen, hydroxy, protected hydroxy, C₁₋₆ alkyloxy, —OC(O)R^{x}, —OCH₂SCH₃ or —OC(O)OR^{x};
one of R^{6b} or R^{7b} is hydrogen and the other is hydroxy, protected hydroxy, C₁₋₆ alkanoyloxy or —OCH₂SCH₃; or
R^{6b} and R^{7b} together form an oxo group; with the proviso that at least one of R^{1b}, R^{2b}, R^{3b}, R^{6b} or R^{7b} is —OCH₂SCH₃;
p is 0 or 1;
R^{x} is a radical of the formula 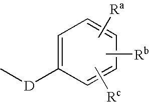
wherein
D is a bond or C₁₋₆ alkyl; and
R^{a}, R^{b} and R^{c} are independently hydrogen, amino C₁₋₆ alkylamino, di-C₁₋₆ alkylamino, halogen, C₁₋₆ alkyl, or C₁₋₆ alkoxy;
R⁴ and R⁵ are independently C₁₋₆ alkyl, C₂₋₆ alkenyl, C₂₋₆alkynyl, or —ZR⁶; wherein
Z is a direct bond, C₁₋₆ alkyl or C₂₋₆ alkenyl; and
R⁶ is aryl, substituted aryl, C₃₋₆ cycloalkyl, or heteroaryl;
b) in a suitable mixture of solvents;
c) in a pharmaceutically effective amount of a buffer, and
d) containing a mixture of antioxidants.

2. The composition in accordance with claim 1, which comprises
a) a pharmaceutically effective amount of a compound of the formula 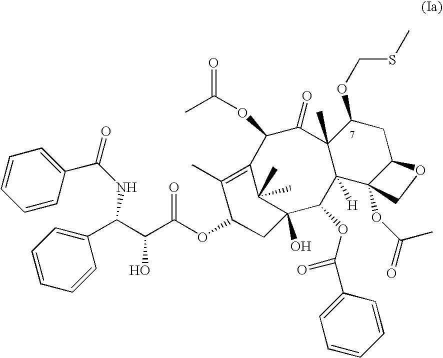
b) in a suitable mixture of solvents;
c) in a pharmaceutically effective amount of a buffer, and
d) containing a mixture of antioxidants.

3. The composition of claim 1 wherein the suitable solvent in step (b) is selected from ethanol, t-butyl alcohol, propylene glycol, glycerin, benzyl benzoate and N,N-dimethylacetamide.

4. The composition of claim 1 wherein the buffer in step (c) is a citrate, tartrate, fumarate, oxalate, benzoate, acetate, succinate or lactate buffer.

5. The composition of claim 1 wherein the co-solvent in step c) is polyoxyethylated (POE) castor oil, polysorbate or polyethylene glycol.

6. The composition of claim 1 wherein the antioxidants in step d) are selected from the group consisting of sodium formaldehye sulfoxylate, ascorbic acid, monthioglycerol, L-cysteine HCl, sodium bisulfite, butylatedhydroxytoluene, propyl gallate and vitamin E.

7. The composition of claim 1 wherein the antioxidants in step (d) are L-cysteine HCl, sodium formaldehyde sulfoxylate and sodium ascorbate.

8. The composition of claim 8 where in the antioxidants are present in about 0.1% w/v.

9. The composition of claim 1 which comprises about 1 μg/mL to about 20 mg/mL of Compound I, about 0.01 to about 0.2 mL/mL of ethanol and about 0.01 to about 0.1 mL/mL of POE castor oil and about 0.01 to about 5 mg/mL of L-cysteine HCl, sodium formaldehyde sulfoxylte and sodium ascorbate.

10. The composition of claim 9 wherein the therapeutic agent is a compound of the formula

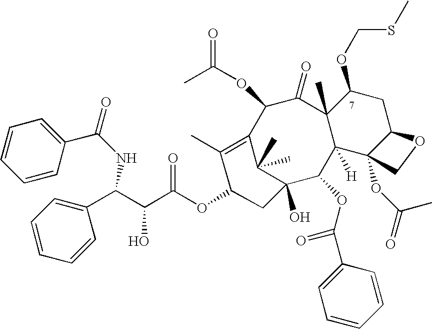

11. The composition of claim 10 comprising 15.0 mg/mL of Compound 1a, 0.075 mL/mL of dehydrated alcohol, 5.16 mg/mL of sodium tartrate dihydrate, 0.04 mg/mL of POE castor oil, 1.11 mg/mL of L-cysteine monohydrate HCl, 1.31 mg/mL of sodium formaldehyde sulfoxylate dihydrate, 1.12 mg/mL of sodium ascorbate and water.

12. The process of claim 11 wherein the composition is administered intravenously.

13. The pharmaceutical composition of claim 1 wherein the composition comprises an antitumor effective amount of the compound of the formula 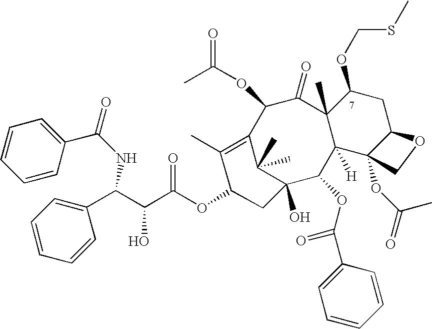
in a pharmaceutically acceptable carrier.

14. A method for inhibiting tumor growth which comprises administering to a patient in need thereof a tumor-growth inhibiting amount of the composition as claimed in claim 1.
